(19) United States
(12) Patent Application Publication
(10) Pub. No.: US 2026/0096943 A1
(43) Pub. Date: Apr. 9, 2026

(54) SKELETAL DISTRACTION DEVICE

(71) Applicant: CORSET ST-FRANCIS INC., Quebec (CA)

(72) Inventors: Jean-François ROY, Quebec (CA); Olivier BOISVERT, Sainte-Marie (CA)

(21) Appl. No.: 19/115,502

(22) PCT Filed: Oct. 20, 2023
(86) PCT No.: PCT/CA2023/051395

§ 371 Date: Mar. 26, 2025

Related U.S. Application Data
(60) Provisional application No. 63/417,738, filed on Oct. 20, 2022.

(51) Int. Cl. A61H 1/02; A61F 5/01; A61F 5/02

(52) U.S. Cl. CPC A61H 1/0218; A61F 5/028; A61H 1/024; A61H 1/0277; A61F 2005/0197; A61H 2201/0192; A61H 2201/1207; A61H 2201/14; A61H 2201/1604; A61H 2201/1614; A61H 2201/1619; A61H 2201/1628; A61H 2201/1635; A61H 2201/164; A61H 2201/165; A61H 2203/03

(57) ABSTRACT

A skeletal distraction device for a four-limbed vertebrate, and particularly a human, comprises a first brace, a second brace and a load transfer mechanism. The first brace is adapted to contact a first body portion of the vertebrate while the second brace is adapted to contact a second body portion of the vertebrate. The load transfer mechanism, which connects the first brace to the second brace, comprises a first spring, a second spring and a preload adjustment. A first spring preload is adjusted through the preload adjuster. The second spring is operative to transfer a second load between the first brace and the second brace along a second axis perpendicular to the first axis. When the vertebrate wears the skeletal distraction device, the first spring is operative to transfer a first load along the first axis between the first brace and the second brace by adjusting the preload.

### FIELD OF THE INVENTION

The present invention generally relates to the field of skeletal decompression devices such as lumbar supports. More specifically, the invention relates to a device adapted for everyday use by people suffering from joint pains, such as chronic back pains. The device works by transferring some of the load taken by one portion of the body on a first side of a joint to a second portion of the body on the other side of the joint. For example, the device may work by transferring to the hips some of the load taken by the upper body and normally supported by the spine, while allowing everyday required mobility.

### BACKGROUND OF THE INVENTION

Prior art exists that is aimed at either medical treatment or at everyday pain relief. For example, European Patent no. EP 319 224 to Stabholz discloses a traction device and lumbar traction apparatus designed for the treatment of low back disorders of disc origin, as well as for the treatment of patients suffering from dorso-lumbar scoliosis and from some vertebral fractures. However, due to its rigid design required for traction of the vertebrae, this traction device is rather suited for medical care when the patient is at rest since it does not offer the required flexibility for everyday use in order to relieve back pain during normal activities.

European Patent no. EP 3 381 417 to Ferrigolo et al. discloses an orthopedic dorsal support brace or orthosis for the backbone, to be used as a support for the backbone of subjects having articular back problems or suffering from osteoporosis. Although the dorsal support brace offers some lateral mobility of the back through a single rotation point, it does not provide much lateral stability nor is it capable of relieving pressure on the vertebrae.

U.S. Pat. No. 4,715,362 to Scott discloses a traction system for use while walking. The traction system is provided with a plurality of telescopic struts. This system is however complex to adjust as many struts must be independently adjusted. Moreover, the level of support provided by all those individual telescopic struts is difficult to adjust uniformly since changing the length of the struts also vary the active length of the springs. Considering that a wearer body is not perfectly symmetrical, different lengths of struts yield different supporting force provided by the springs of those struts.

U.S. Pat. No. 5,916,188 to Ousdal discloses a device for stretching the body, more particularly the torso. This device uses pneumatic telescopic struts which are connected through pivots. Such struts do not follow the natural flexing or bending movements of the human body and therefore may lead to a lack of comfort when worn.

There is therefore a need for a skeletal distraction device that overcomes or mitigates one or more disadvantages of known similar devices.

### SUMMARY OF THE INVENTION

It is an object of the present invention to provide a skeletal distraction device that overcomes or mitigates one or more disadvantages of known similar skeletal distraction devices for different limbs such as lumbar traction devices, decompression devices and other back supports, or at least provides a useful alternative.

The invention provides the advantages of being convenient to use, and simple to adjust. The device also follows the natural movements of a vertebrate, and in particular a human, so that the muscles of the vertebrate keep on being solicited while the device is worn. This advantageously prevents muscle atrophy while reaching the objective of reducing a load on an articulation to be relieved.

In accordance with an embodiment of the present invention, there is provided a skeletal distraction device for a four-limbed vertebrate comprising a first brace, a second brace and a load transfer mechanism. The first brace is adapted to contact a first body portion of the vertebrate while the second brace is adapted to contact a second body portion of the vertebrate. The first body portion and the second body portion are connected through at least one joint permitting a relative movement between the first body portion and the second body portion. The load transfer mechanism, which connects the first brace to the second brace, comprises a first spring, a second spring and a preload adjustment. The first spring is connected to the first brace at a first end. The first spring has a first axis. The preload adjuster is connected to the second brace and to a second end of the first spring. The preload adjuster is operable to adjust a preload of the first spring when the first brace and the second brace are respectively in contact with the first body portion and the second body portion of the vertebrate. The second spring is connected to the first brace at a first extremity and to the second brace at a second extremity. The second spring is operative to transfer a second load between the first brace and the second brace along a second axis perpendicular to the first axis. When the vertebrate wears the skeletal distraction device, the first spring is operative to transfer a first load along the first axis between the first brace and the second brace by adjusting the preload.

The second spring may slidingly engage one of the first and the second brace at its respective one of the first extremity and the second extremity. The second spring may be fixedly connected to the first brace and slidingly connected to the second brace.

The first spring may have coils and the second spring may be straight when unloaded. The first axis passes through centers of the coils.

The first spring may have a first spring constant along the first axis and the second spring may have a second spring constant along the second axis. The first spring constant and the second spring constant may be different from each other.

The second spring may be positioned within the coils of the first spring.

The second spring may be made of a composite material.

The preload adjuster may comprise a slider connected to the first spring at the second end. The slider is operable to displace the second end of the first spring along the first axis.

The preload adjuster may comprise a ratcheting mechanism preventing the slider from retracting under the preload.

The preload adjuster may comprise an adjusting knob or a motor which are connected to the slider and which are operative to move the slider in order to adjust the preload of the first spring.

The second spring may be slideable with respect to the slider and is devoid of load bearing capacity along the first axis.

The load transfer mechanism may comprise a left load transfer mechanism and a right load transfer mechanism. The left load transfer mechanism connects a left portion of the first brace to a left portion of the second brace and the right load transfer mechanism connects a right portion of the first brace to a right portion of the second brace.

The first springs and the second springs of respective the left and right load transfer mechanisms may be adapted to be substantially aligned with a frontal plane of the vertebrate when in use.

Each one of the first brace and of the second brace may comprise a size adjusting mechanism operable to adjust a size of the respective first brace and second brace so as to snugly fit a respective body portion of the vertebrate.

In particular, the skeletal distraction device may be one of:

a vertebral distraction device. In this case the first brace is a torso brace and the second brace is a pelvic brace. The torso brace is adapted to contact an upper portion of a torso of the vertebrate and the pelvic brace is adapted to rest on a pelvic region of the vertebrate;

a knee distraction device. In this case, the first brace is a femur brace and the second brace is a tibia brace. The femur brace is adapted to contact a femur portion of a leg of the vertebrate and the tibia brace is adapted to contact a tibia portion of the leg of the vertebrate;

an elbow distraction device. In this case, the first brace is a radius brace and the second brace is a humerus brace. The radius brace is adapted to contact a radius portion of an arm of the vertebrate and the humerus brace is adapted to contact a humerus portion of the arm of the vertebrate; and a cervical distraction device. In this case, the first brace is a skull brace and the second brace is a shoulder brace. The skull brace is adapted to contact a skull of the vertebrate and the shoulder brace is adapted to rest on shoulders of the vertebrate.

The first brace, the second brace and the load transfer mechanism may be adapted to a physiology and weight of the vertebrate, which can be selected from the group consisting of a human, a vertebrate domestic animal, a vertebrate farm animal and a vertebrate wild animal.

In accordance with another embodiment of the present invention, there is provided a vertebral distraction device as defined above. The torso brace may comprise a left torso brace portion, a right torso brace portion and a torso strap. The torso strap connects the left torso brace portion to the right torso brace portion. The torso strap is adjustable so as to be operative to workably fit the right torso brace portion and the left torso brace portion on an upper portion of the torso.

The torso brace may be adapted to contact the upper portion of the torso in a lower region of a rib cage of the vertebrate. The torso strap is adapted to contact the vertebrate under a breast region of the vertebrate.

The pelvic brace may comprise a left pelvic brace portion, a right pelvic brace portion and a pelvic strap. The pelvic strap connects the left pelvic brace portion to the right pelvic brace portion and is adjustable so as to be operable to adjust the right pelvic brace portion and the left pelvic brace portion on a pelvis of the pelvic region of the vertebrate.

In accordance with another embodiment of the invention, there is provided a vertebral distraction device for a human wearer having a frontal axis, a longitudinal axis and a sagittal axis. The vertebral distraction device comprises a torso brace, a pelvic brace and a load transfer mechanism. The torso brace is adapted to contact an upper portion of a torso of the wearer. The pelvic brace is adapted to rest on a pelvic region of the wearer. The load transfer mechanism connects the torso brace to the pelvic brace. The load transfer mechanism has a first spring, a second spring and a preload adjuster. The first spring connects to one of the torso brace and the pelvic brace at a first end. The preload adjuster connects to one other of the torso brace and the pelvic brace. The preload adjuster is connected to a second end of the first spring and is operable to adjust a preload of the first spring along the longitudinal axis when the torso brace and the pelvic brace are respectively in contact with the torso and the pelvic region of the wearer. The second spring is fixedly connected to one of the torso brace and the pelvic brace at a first extremity and, at a second extremity, slidingly engages the other one of the torso brace and the pelvic brace along the longitudinal axis. When the wearer wears the vertebral distraction device, the first spring is operative to transfer a first load along the longitudinal axis between the torso brace and the pelvic brace by adjusting the preload while the second spring is operative to support a second load along at least one of the frontal axis and the sagittal axis. The second spring remains slideable at the second extremity with respect to the other one of the torso brace and the pelvic brace.

### BRIEF DESCRIPTION OF DRAWINGS

These and other features of the present invention will become more apparent from the following description in which reference is made to the appended drawings wherein:

FIG. 1 is a perspective view of a human showing a reference system of planes and axes;

FIG. 2 is front view of the human of FIG. 1 wearing a skeletal distraction device in accordance with an embodiment the present invention;

FIG. 3 is a perspective view of the skeletal distraction device of FIG. 2;

FIG. 4A is perspective view of a left side only of the skeletal distraction device of FIG. 2 with a cover removed to show an interior of a manually operated preload adjustment mechanism;

FIG. 4B is a detail perspective view of the interior of the manually operated preload adjustment mechanism of FIG. 4A;

FIG. 5 is a front view of the human of FIG. 2 bending its torso laterally, i.e. around the frontal axis;

FIG. 6 is a side view of the human of FIG. 2 bending its torso forward, i.e. around the sagittal axis;

FIG. 7 is a side view of a left side of the skeletal distraction device of FIG. 2 showing the interior of the manually operated preload adjustment mechanism with its slider in an extended position;

FIG. 8 is a rear view of a motorized load transfer mechanism in accordance with an embodiment of the present invention;

FIG. 9 is a cut-away side view of the motorized load transfer mechanism of FIG. 8;

FIG. 10 is an exploded perspective view of the motorized load transfer mechanism of FIG. 8;

FIG. 11 is a top view of a variant of a deployed torso brace and pelvic brace in accordance with an embodiment of the invention;

FIG. 12 is a top view of another variant of a deployed torso brace and pelvic brace in accordance with an embodiment of the invention;

FIG. 13a is a side view of a human wearing a cervical distraction device in accordance with an embodiment of the invention;

FIG. 13b is a front view of the human of FIG. 13a wearing the cervical distraction device of FIG. 13a;

FIG. 14a is a side view of a human wearing a knee distraction device in a straight leg position in accordance with an embodiment of the invention;

FIG. 14b is a front view of the human of FIG. 14a wearing the knee distraction device of FIG. 14a; and

FIG. 14c is a side view of the human of FIG. 14a wearing the knee distraction device of FIG. 14a in a bent leg position.

### DETAILED DESCRIPTION OF THE INVENTION

The present invention relates to a skeletal distraction device capable of providing skeletal distraction to its wearer. Adapted to any four-limbed vertebrate such as domestic animals, farm animals or wild animals, but more particularly adapted to humans, the skeletal distraction device of the present invention is intended to contribute to providing relief to body portions connected through at least one joint permitting a relative movement between the body portions.

For example, the skeletal distraction device may be a vertebral distraction device, a knee distraction device, an elbow distraction device or a cervical distraction device, or their equivalent for animals. To better illustrate the present invention, a non-restrictive example will be provided with a vertebral distraction device for humans. The vertebral, or spinal, distraction reduces the internal pressure of the intervertebral disc, increases the space between the vertebrae and reduces the pressure on the nerve roots, thereby alleviating back pain for the human wearer.

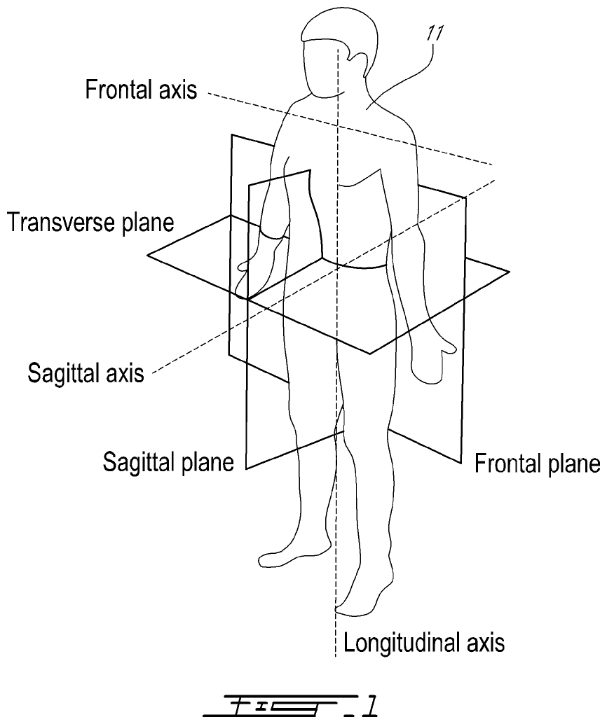

For reference purposes, FIG. 1 depicts standard axis and planes of the human body, to which reference will later be made in the description.

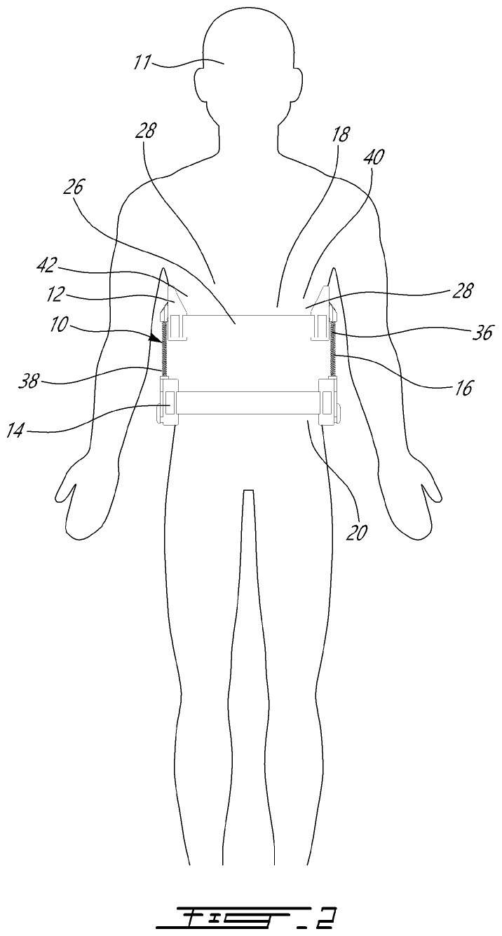

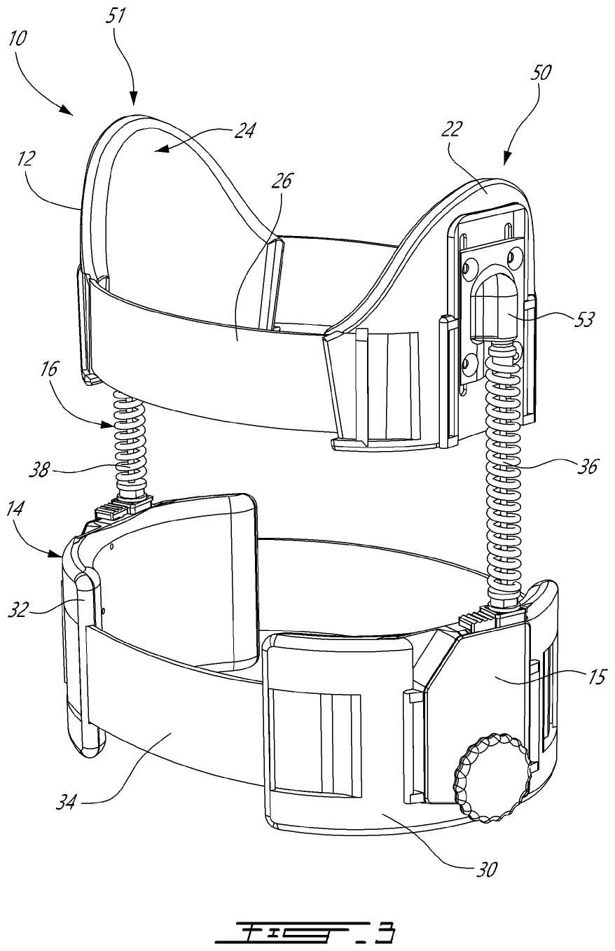

FIG. 2, now concurrently referred to, depicts a skeletal distraction device, and more particularly a vertebral distraction device 10 for a wearer 11. The vertebral distraction device 10 comprises a torso brace 12, a pelvic brace 14 and a load transfer mechanism 16. The torso brace 12 is designed to contact an upper portion of a torso 18 of the wearer 11. The pelvic brace 14 is designed to rest on a pelvic region 20 of the wearer 11. The load transfer mechanism 16 connects the torso brace 12 to the pelvic brace 14. The load transfer mechanism 16 attaches to the torso brace 12 through an adaptor plate 53 and to the pelvic brace 14 through a housing 15, best shown in FIG. 3, now concurrently referred to. Conveniently, both the torso brace 12 and the pelvic brace 14 each have at least one surface covered with a soft material to provide increased comfort to the wearer 11.

Typically, the torso brace 12 is made of two halves, that is a left torso brace 22 and a right torso brace 24 which are interconnected by a torso adjuster 26. The torso adjuster 26 is adjustable to allow the torso brace 12 to workably fit on the upper portion of the torso 18 by pulling together the right torso brace 22 and the left torso brace 24. The torso brace 12 is adapted to contact the upper portion of the torso 18 in a lower region of a rib cage 28 of the wearer 11. The torso adjuster 26 is adapted to contact the wearer 11 under a breast region 28 of the wearer 11. This advantageously allows having a single model of vertebral distraction device 10 which fits both women and men (although different sizes may be provided).

Similarly, the pelvic brace 14 may comprise two halves, that is a left pelvic brace 30 and a right pelvic brace 32 which are interconnected by a pelvic adjuster 34. The pelvic adjuster 34 is also adjustable so as to operably adjust the right pelvic brace 32 and the left pelvic brace 30 on a pelvis of the pelvic region 20 of the wearer 11 by adjusting the distance between both the left and the right pelvic braces 30, 32.

As the torso brace 12 and the pelvic brace 14 are typically made of two halves, the load transfer mechanism 16 similarly comprises a left load transfer mechanism 36 and a right load transfer mechanism 38. The left and the right load transfer mechanisms 36, 38 are respectively connected the left and the right torso braces 22, 24 to the left and the right pelvic braces 30, 32. The left and the right load transfer mechanisms 36, 38 are typically located on an exterior of their respective left and right torso braces 22, 24 and left and right pelvic braces 30, 32 so that the left and right load transfer mechanisms 36, 38 are respectively located on a left and right side 40, 42 of the torso 18 of the wearer 11 when in use. This advantageously provides some clearance with the wearer's torso when the wearer moves around.

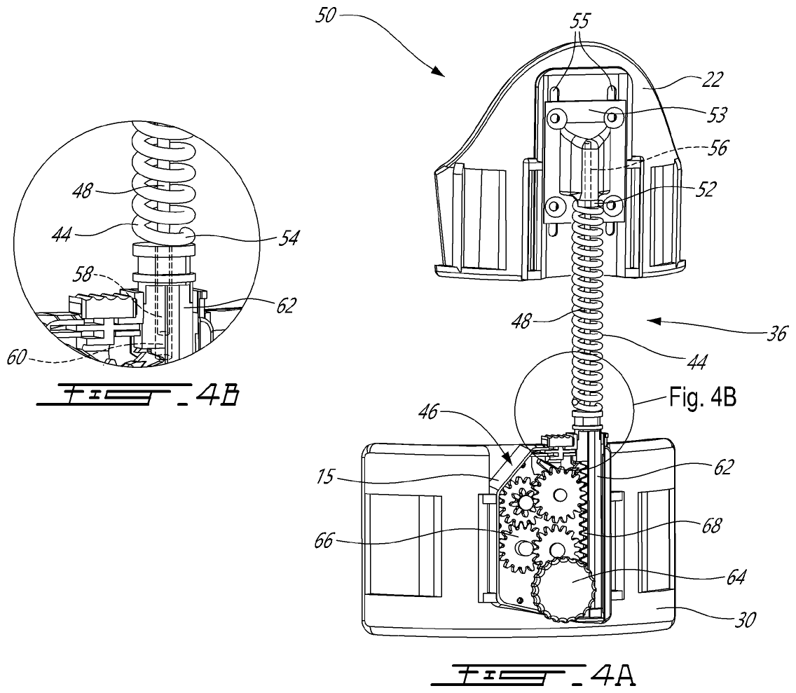

FIG. 4A, now concurrently referred to, depicts a left half 50 of the vertebral distraction device 10. For simplicity, only the left half 50 will be described, the right half 51 of the vertebral distraction device 10 being essentially a mirror image of the left half 50, comprising the same elements and working the same way. The left load transfer mechanism 36 comprises a first spring 44, a preload adjustment mechanism 46 and a second spring 48. This assembly could be reversed and the first end 52 of the first spring 44 could rather be connected to the left pelvic brace 30 and the preload adjustment mechanism 46 could be located on the left torso brace 22. Although such an arrangement would work, it would not be as convenient to adjust the preload adjustment mechanism 46.

The first spring 44 is connected to the left torso brace 22 at a first end 52. The attachment position of the first end 52 to the torso brace 22 is adjustable along the longitudinal axis by sliding the adaptor plate 53 through adjustment rails 55 and locking it in place with screws (not shown) inserted in chamfered holes 57 and tightened.

In the figures, the first spring 44 is depicted as a coil spring. However, this should not be interpreted restrictively since other types of suitable springs, such as a resilient rubber for example, could be used. Also, the first spring 44 has a first spring constant along the longitudinal axis. The first spring constant is independent of a distance between the torso brace 12 and the pelvic brace 14 since all the coils of the first spring 44 remain active over the whole length of the first spring 44.

The left preload adjustment mechanism 46 is conveniently located on the left pelvic brace 30 and is connected to a second end 54 of the first spring 44. The preload adjustment mechanism 46 is operative to adjust a preload of the first spring 44 along the longitudinal axis when the left torso brace 22 and the left pelvic brace 30 are respectively in contact with the upper portion of the torso 18 and the pelvic region 20 of the wearer 11. The second spring 48 is connected to the left torso brace 22 through a fixed connection (e.i. clamped, e.i. no degrees of freedom) at a first extremity 56. At a second extremity 58, the second spring 48 slidingly engages the left pelvic brace 30 along the longitudinal axis through a cavity 60 in a slider 62 and is held tightly in the cavity 60, thereby providing a single degree of freedom to the second extremity 58 along the longitudinal axis, i.e. along a length of the cavity 60. This is best shown in FIG. 4B, now concurrently referred to. This sliding engagement of the second spring 48 allows for independent adjustment of the distance between the left torso brace 22 and the left pelvic brace 30 and as well allows accommodating natural movements of the wearer 11, for instance when the wearer 11 bends its torso towards the left or the right, or bends forward. This sliding engagement of the second spring 48 in the slider 62 also means that the second spring 48 is totally devoid of load bearing capacity along the longitudinal axis. In other words, the load transferred from the torso brace 12 to the pelvic brace 14 is only transferred through the first springs 44.

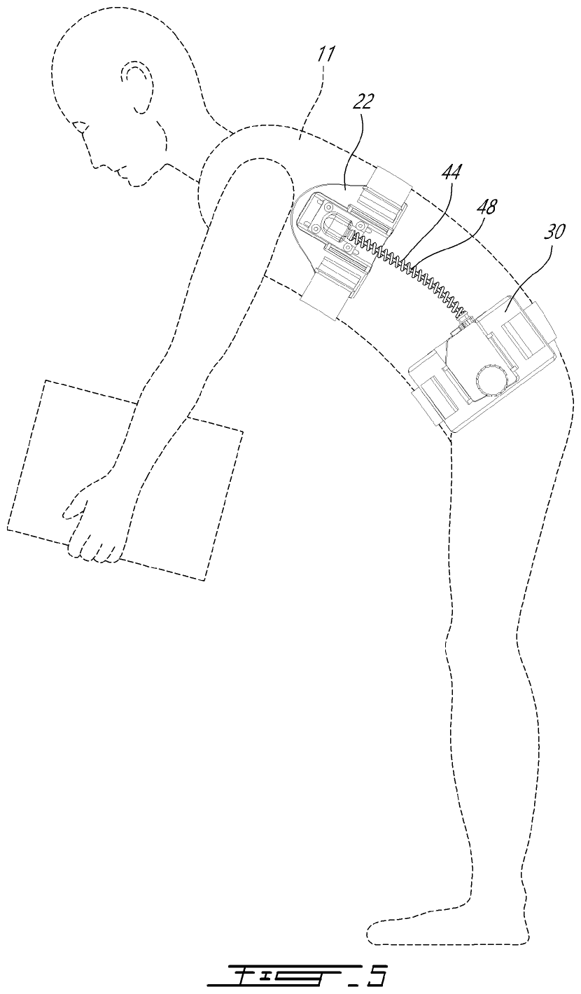

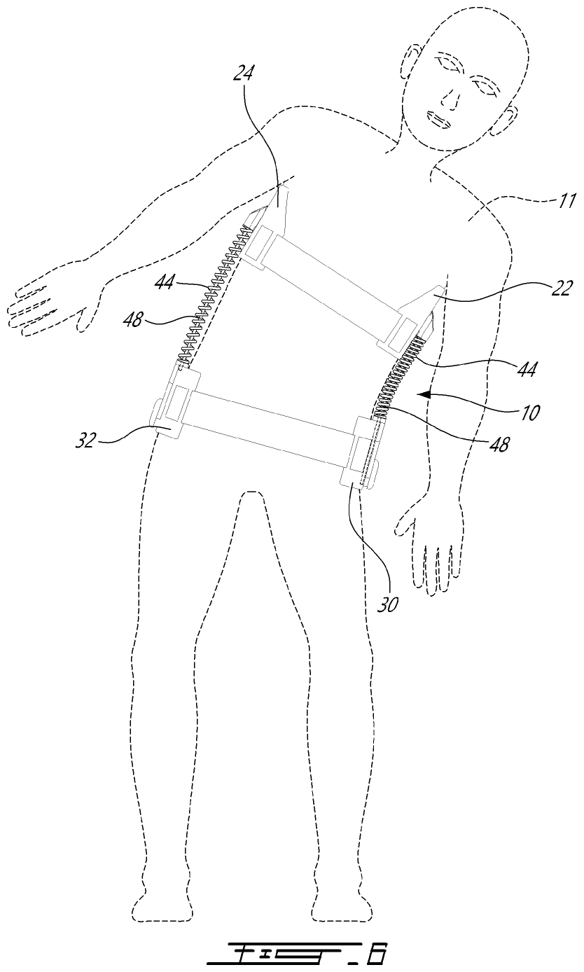

The second spring 48 basically acts as a leaf spring between the torso brace 12 and the pelvic brace 14 and creates reacting forces when the wearer 11 either bends its torso around the sagittal axis, as depicted in FIG. 5, or around the frontal axes as depicted in FIG. 6. Both FIGS. 5 and 6 are now concurrently referred to. Although the second spring 48 is depicted as a rod, having a circular cross-section and thereby having the same spring constant in bending around the sagittal axis and around the frontal axis, the second spring 48 could adopt different cross-sections depending on its required characteristics. For example, the second spring 48 could have an elliptic cross-section with its long axis oriented along the sagittal axis and its short axis aligned along the frontal axis. This way, the wearer 11 bending its torso around the frontal axis would have the second spring 48 generate a larger reaction force than when the wearer 11 bends around the sagittal axis. In other words, the support provided by the second spring 48 can be adapted to the needs and situation of each wearer 11 by selecting the size and the shape of the cross-section of the second spring 48. Of course, the cavity 60 must then adopt a matching cross-section to restrict the movements of the secondary spring 48 in all degrees of freedom except the longitudinal axis. The second spring 48 is typically made of steel, plastic or of a composite material such as glass or carbon fiber. In the figures, the second spring 48 is shown positioned within the first spring 44, but it does not have to be. Installing the second spring 48 within the first spring 44 is convenient as it requires less space. Typically, the first spring 44 and the second spring 48 of the respective left and right load transfer mechanism 36, 38 may be substantially aligned with a frontal plane when the vertebral distraction device 10 is worn by the wearer 11 and when the wearer 11 is standing up.

Because the second spring 48 has its first extremity 56 clamped and its second extremity 58 clamped as well except along the longitudinal axis, the second spring 48 acts as a leaf spring, providing a regular and continuous arc of the second spring 48, and providing thereby a more natural positioning of both the torso brace 12 and the pelvic brace 14 with respect to each other. In turn, because of their continuous curvature, both second springs 48 allow an improved spinal distraction when the wearer 11 bends his/her torso around the frontal axis, such as in FIG. 5. Inducing such a spinal distraction reduces the internal pressure of the intervertebral disc, increases the space between the vertebrae and thereby reduces the pressure on the nerve roots. In a similar fashion, when the wearer 11 bends his/her torso to the side (i.e. around the sagittal axis) as in FIG. 6, the continuous, arcuate shape taken by both secondary springs 48 substantially follows the shape of the torso and the spine of the wearer 11. This allows a natural support of the torso and a clearance of the torso on both sides (i.e. prevents pinching the torso on an exterior side of the torso). Moreover, the natural curvature of the second springs 48 allows guiding the first springs 44 along a similar curve since the left and right second springs 48 are located within the respective left and right first springs 44.

FIG. 7 is concurrently referred to. The preload adjustment mechanism 46 is equipped with an adjusting knob 64 connected to the slider 62 through a train of gears 66 and a rack 68 on the slider 62. Through turning the adjusting knob 64 one way or the other moves the slider 62 along the longitudinal axis. If the vertebral distraction device 10 is not worn, that is if the vertebral distraction device is in a free state, turning the adjustment knob 64 varies the distance between the left torso brace 22 and the left pelvic brace 30. On the other hand, if the vertebral distraction device 10 is properly worn by the wearer 11 and fixed in place firmly through their respective torso adjuster 26 and pelvic adjuster 34, turning the adjustment knob 64 compresses the first spring 44 and creates a preload in this first spring 44. This is the case depicted in FIG. 7. This preload is equal to half the weight that will be transferred to the legs of the wearer 11 through the pelvic brace 14, assuming that both the left and the right preload adjustment mechanisms 46 are similarly adjusted and that both transfer the same half-load to their respective left and right pelvic braces 30, 32. In other words, the pelvic brace 14 transfers the sum of the preloads induced by the left and right preload adjustment mechanisms 46 in the respective left and right first springs 44. For example, if the preload is of 50 N on each first spring 44, then a total force of 100 N is transferred to the pelvic region 20 and represents as much force that does not have to be supported by the wearer's spine when the wearer 11 is standing up. Since F=mg along a vertical axis, a force of 100 N represents a weight of 10.2 kg or 22.5 lbs. Assuming for a moment that each first spring 44 has a spring constant of 1 N/mm, this means that each first spring 44 must be compressed by 50 mm (2.26 in) by their respective slider 62 to each generate a force of 50 N.

To prevent the slider 62 from retracting under the preload and to prevent overloading the train of gears 66, the preload adjustment mechanism 46 is also equipped with a ratcheting mechanism 70 which can be locked and unlocked with a sliding button 72. It may be noted that one gear from the train of gears 66 has been hidden in FIG. 7 to expose the ratcheting mechanism 70.

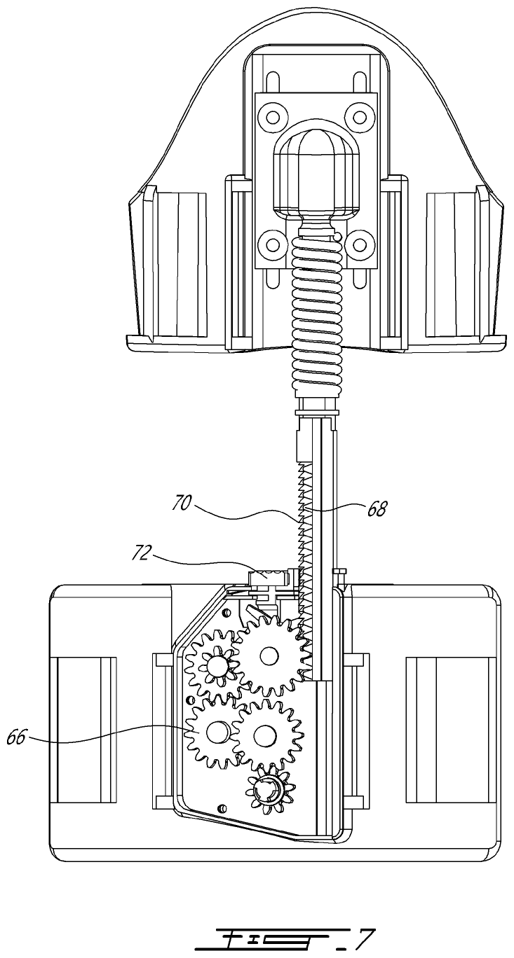

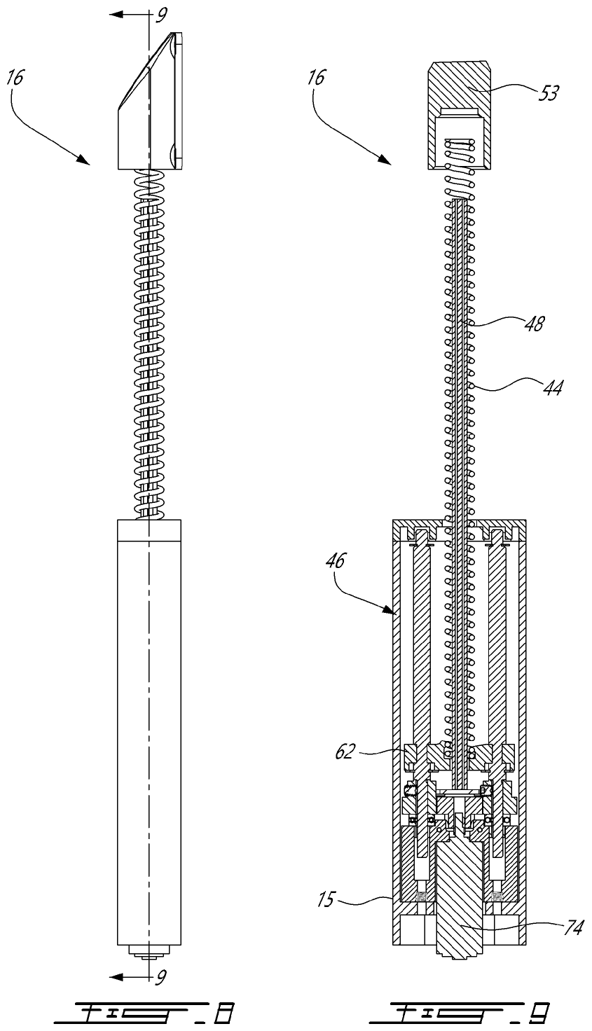

Alternatively to manual adjustment with the adjusting knob 64 and the train of gears 66, a compact electric motor 74 connected to a switch and a battery may be used. This embodiment is represented in FIGS. 8 to 10, now concurrently referred to. The mode of operation of the preload adjustment is exactly the same as previously described, except that the electric motor 74 is operated through the switch, the electric motor 74 being operable to displace the slider 62 the same as may be done manually through the adjusting knob 64. In fact, the exact assembly of the motorized version of the load transfer mechanism 16 may vary slightly from that of the manual version and as depicted in FIGS. 9 and 10. Different variants of mechanisms may be designed to adequately adjust the preload of the first spring 44.

In use, the wearer 11 adjusts the pelvic brace 14 on its pelvis by adjusting the pelvic adjuster 34 so that the left and right pelvic braces 30, 32 sit closely but comfortably on the pelvis. Similarly, the wearer 11 then adjust the torso brace 12 on the upper portion of the torso 18 using the torso adjuster 26 in order to make the left and right torso braces 22, 24 fit closely against, namely, a lower portion of the rib cage. Once both the torso brace 12 and the pelvic brace 14 are installed, the wearer 11, reaches the preload adjustment mechanisms 46 on each one of the left and right load transfer mechanisms 36, 38. The wearer 11 adjusts the compression of the left and right first springs 44 by turning the adjusting knobs 64 so as to extend the sliders 62 of both the left and right load transfer mechanisms 36, 38 until the right amount of support by the first springs 44 is reached. So worn, the left and right second springs 48 are operative to support a second load induced by movement of the wearer 11 around at least one of the frontal axis and the sagittal axis.

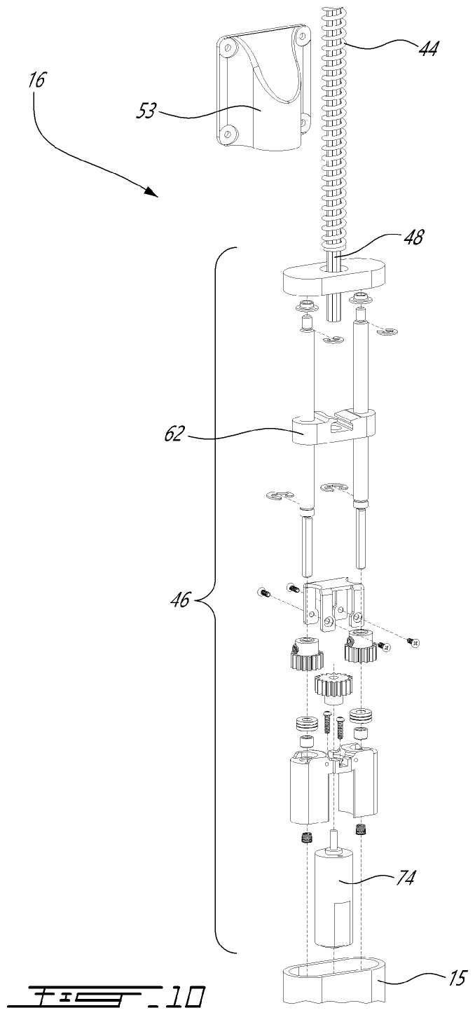

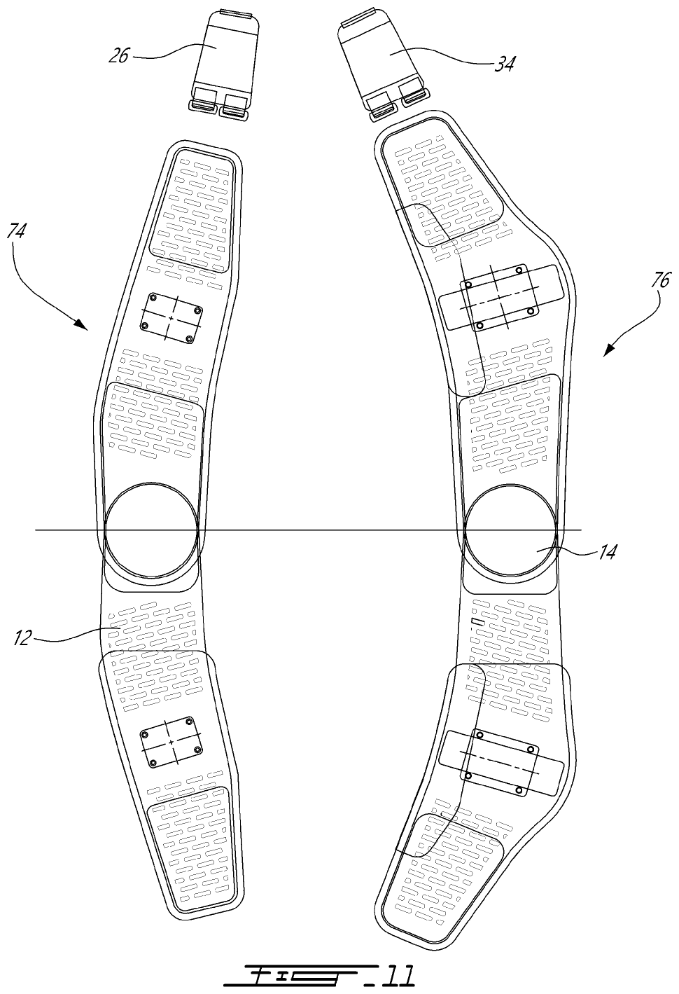

FIGS. 11 and 12 show examples of flat patterns 76 and 78 of respectively the torso brace 12 and the pelvic brace 14. The torso brace 12 is equipped with the torso adjuster 26 for precise adjustment over the torso of the wearer 11. Similarly, the pelvic brace 14 is equipped with the pelvic adjuster 34 for precise adjustment over the torso of the wearer 11.

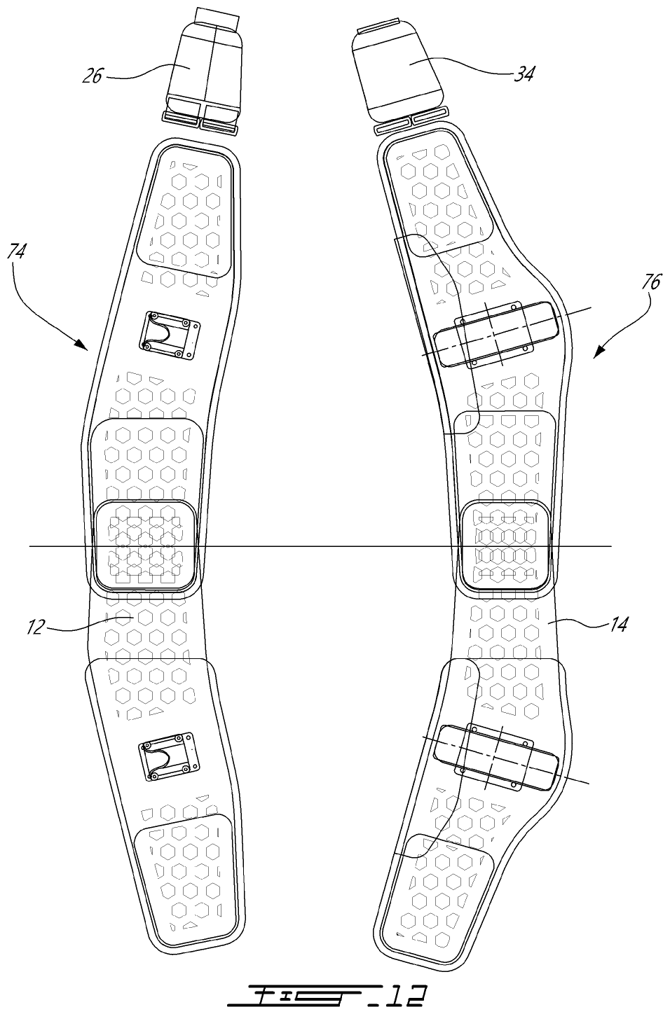

FIGS. 13a and 13b, now concurrently referred to, depict another embodiment of the present invention. More precisely, the skeletal distraction device 10 represented is a cervical distraction device 80 for a human wearer 11. In this case, a shoulder brace 82 is designed to rest on the shoulders of the wearer 11 while a skull brace 84 supports the head of the wearer 11 in the occipital bone region. Similarly to the vertebral distraction device described previously, two load transfer mechanisms 16 interconnect the shoulder brace 82 to the skull brace 84 in order to distract the spine of the wearer 11 in the cervical region. These two load transfer mechanisms 16 allow adjusting the preload on the first spring 44 in order to distract the spine of the wearer 11 in the cervical region.

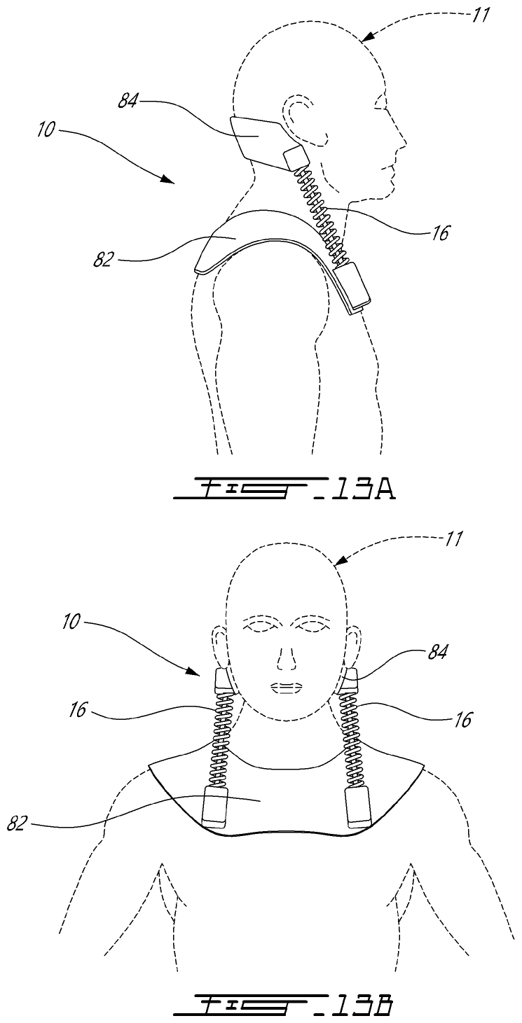

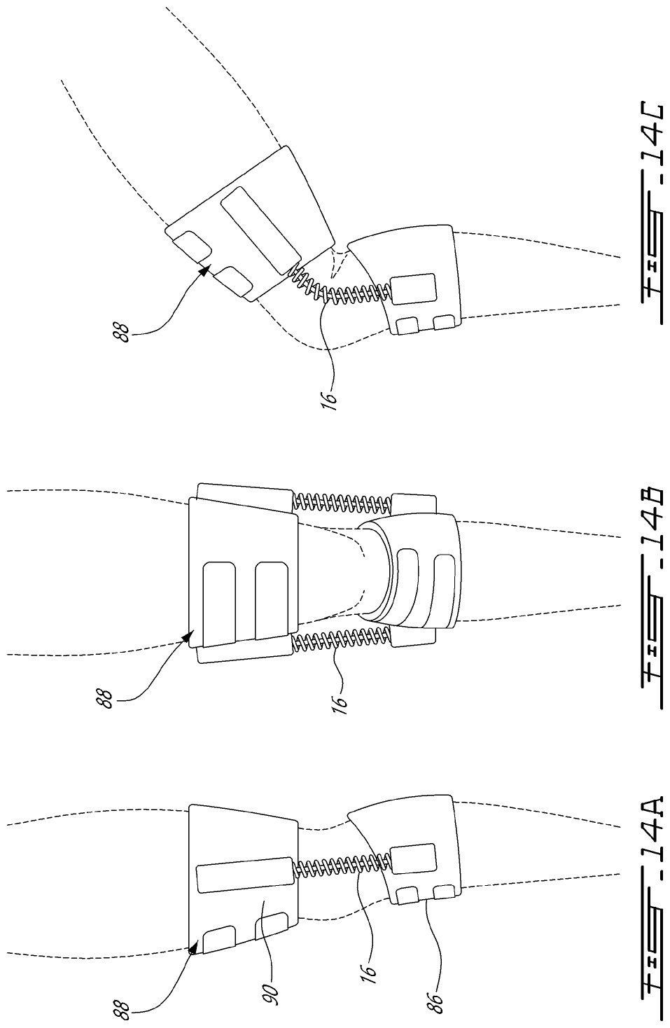

FIGS. 14a, 14b and 14c, now concurrently referred to, depict yet another embodiment of the present invention. More precisely, the skeletal distraction device 10 represented is a knee distraction device 86 for a human wearer 11. In this case, a tibia brace 86 is designed to rest snugly on a calf region of the lower leg, right below the knee of the wearer 11 while a femur brace 88 snugly fits around the thigh the wearer 11. Similarly to the vertebral distraction device described previously, two load transfer mechanisms 16 interconnect the tibia brace 86 to the femur brace 88. These two load transfer mechanisms 16 allow adjusting the preload on the first spring 44 in order to distract the knee joint of the wearer 11.

The elbow being a very similar type of joint to the knee, an elbow distracting device similarly comprises a radius brace and a humerus brace interconnected by the load transfer mechanism. The radius brace is adapted to contact a radius portion of an arm of the human wearer and the humerus brace is adapted to contact a humerus portion of the arm.

The present invention has been described with regard to preferred embodiments. The description as much as the drawings were intended to help the understanding of the invention, rather than to limit its scope. It will be apparent to one skilled in the art that various modifications may be made to the invention without departing from the scope of the invention as described herein, and such modifications are intended to be covered by the present description. The invention is defined by the claims that follow.

What is claimed is:

1. A skeletal distraction device for a four-limbed vertebrate comprising:
a first brace adapted to contact a first body portion of the vertebrate;
a second brace adapted to contact a second body portion of the vertebrate, the first body portion and the second body portion being connected through at least one joint permitting a relative movement between the first body portion and the second body portion; and
a load transfer mechanism connecting the first brace to the second brace, the load transfer mechanism having:
a first spring connected to the first brace at a first end, the first spring having a first axis;
a preload adjuster connected to the second brace, the preload adjuster being connected to a second end of the first spring and being operable to adjust a preload of the first spring when the first brace and the second brace are respectively in contact with the first body portion and the second body portion of the vertebrate;
a second spring connected to the first brace at a first extremity and to the second brace at a second extremity, the second spring being operative to transfer a second load between the first brace and the second brace along a second axis perpendicular to the first axis;
wherein when the vertebrate wears the skeletal distraction device, the first spring is operative to transfer a first load along the first axis between the first brace and the second brace by adjusting the preload.

2. The skeletal distraction device of claim 1 wherein the second spring slidingly engages one of the first and the second brace at its respective one of the first extremity and the second extremity.

3. The skeletal distraction device of claim 1 or 2, wherein the second spring is fixedly connected to the first brace and slidingly connected to the second brace.

4. The skeletal distraction device of any one of claims 1 to 3, wherein the first spring has coils and wherein the second spring is straight when unloaded, the first axis passing through centers of the coils.

5. The skeletal distraction device of claim 4, wherein the first spring has a first spring constant along the first axis and wherein the second spring has a second spring constant along the second axis, the first spring constant and the second spring constant being different from each other.

6. The skeletal distraction device of claim 4 or 5, wherein the second spring is positioned within the coils of the first spring.

7. The skeletal distraction device of any one of claims 1 to 6, wherein the second spring is made of a composite material.

8. The skeletal distraction device of any one of claims 4 to 7, wherein the preload adjuster comprises a slider connected to the first spring at the second end, the slider being operable to displace the second end of the first spring along the first axis.

9. The skeletal distraction device of claim 8, wherein the preload adjuster comprises a ratcheting mechanism preventing the slider from retracting under the preload.

10. The skeletal distraction device of claims 8 or 9, wherein the preload adjuster comprises one of an adjusting knob and a motor connected to the slider and operative to move the slider to adjust the preload of the first spring.

11. The skeletal distraction device of claim 10, wherein the second spring is slideable with respect to the slider and is devoid of load bearing capacity along the first axis.

12. The skeletal distraction device of any one of claims 1 to 11, wherein the load transfer mechanism comprises a left load transfer mechanism and a right load transfer mechanism, the left load transfer mechanism connecting a left portion of the first brace to a left portion of the second brace and the right load transfer mechanism connecting a right portion of the first brace to a right portion of the second brace.

13. The skeletal distraction device of claim 12, wherein the first springs and the second springs of respective the left and right load transfer mechanisms are adapted to be substantially aligned with a frontal plane of the vertebrate when in use.

14. The skeletal distraction device of any one of claims 1 to 13, wherein each one of the first brace and of the second brace comprise a size adjusting mechanism operable to adjust a size of the respective first brace and second brace so as to snugly fit a respective body portion of the vertebrate.

15. The skeletal distraction device of any one of claims 1 to 14, wherein the skeletal distraction device is one of:
a vertebral distraction device, the first brace being a torso brace and the second brace being a pelvic brace, the torso brace being adapted to contact an upper portion of a torso of the vertebrate and the pelvic brace being adapted to rest on a pelvic region of the vertebrate;
a knee distraction device, the first brace being a femur brace and the second brace being a tibia brace, the femur brace being adapted to contact a femur portion of a leg of the vertebrate and the tibia brace being adapted to contact a tibia portion of the leg of the vertebrate;
an elbow distraction device, the first brace being a radius brace and the second brace being a humerus brace, the radius brace being adapted to contact a radius portion of an arm of the vertebrate and the humerus brace being adapted to contact a humerus portion of the arm of the vertebrate; and
a cervical distraction device, the first brace being a skull brace and the second brace being a shoulder brace, the skull brace being adapted to contact a skull of the vertebrate and the shoulder brace being adapted to rest on shoulders of the vertebrate.

16. The skeletal distraction device as defined in any one of claims 1 to 15, wherein the first brace, the second brace and the load transfer mechanism are adapted to a physiology and weight of the vertebrate selected from the group consisting of a human, a vertebrate domestic animal, a vertebrate farm animal and vertebrate wild animal.

17. A vertebral distraction device as defined in claim 15, wherein the torso brace comprises a left torso brace portion, a right torso brace portion and a torso strap, the torso strap connecting the left torso brace portion to the right torso brace portion, the torso strap being adjustable so as to be operative to workably fit the right torso brace portion and the left torso brace portion on an upper portion of the torso.

18. The vertebral distraction device of claim 17, wherein the torso brace is adapted to contact the upper portion of the torso in a lower region of a rib cage of the vertebrate, the torso strap being adapted to contact the vertebrate under a breast region of the vertebrate.

19. The vertebral distraction device of claim 17 or 18, wherein the pelvic brace comprises a left pelvic brace portion, a right pelvic brace portion and a pelvic strap, the pelvic strap connecting the left pelvic brace portion to the right pelvic brace portion, the pelvic strap being adjustable so as to be operative to adjust the right pelvic brace portion and the left pelvic brace portion on a pelvis of the pelvic region of the vertebrate.

20. A vertebral distraction device for a human wearer having a frontal axis, a longitudinal axis and a sagittal axis, the vertebral distraction device comprising:
a torso brace adapted to contact an upper portion of a torso of the wearer;
a pelvic brace adapted to rest on a pelvic region of the wearer; and
a load transfer mechanism connecting the torso brace to the pelvic brace, the load transfer mechanism having:
a first spring connected to one of the torso brace and the pelvic brace at a first end;
a preload adjuster connected to one other of the torso brace and the pelvic brace, the preload adjuster being connected to a second end of the first spring and being operative to adjust a preload of the first spring along the longitudinal axis when the torso brace and the pelvic brace are respectively in contact with the torso and the pelvic region of the wearer;
a second spring fixedly connected to one of the torso brace and the pelvic brace at a first extremity and, at a second extremity, slidingly engaging the other one of the torso brace and the pelvic brace along the longitudinal axis;
wherein when the wearer wears the vertebral distraction device, the first spring is operative to transfer a first load along the longitudinal axis between the torso brace and the pelvic brace by adjusting the preload, and the second spring is operative to support a second load along at least one of the frontal axis and the sagittal axis, the second spring still being slideable at the second extremity with respect to the other one of the torso brace and the pelvic brace.
